(19) United States
(12) Patent Application Publication
(10) Pub. No.: US 2004/0024295 A1
(43) Pub. Date: Feb. 5, 2004

(54) System, method and computer program product for measuring blood properties form a spectral image

(76) Inventors: Christopher A Cook, New York, NY (US); Mustafa K Emresoy, Philadelphia, PA (US); Hany Farid, Hanover, NH (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/362,645

(22) PCT Filed: Aug. 27, 2001
(86) PCT No.: PCT/US01/26651

(51) Int. Cl. A61B 5/00

(52) U.S. Cl. 600/310

(57) ABSTRACT

A system, method and computer program product is provided for analyzing spectral images of a micrcirculatory system to measure the volume and concentration of a blood vessel. The images are analyzed to identify vessel structure, measure the light absorption and develop a contrast gradient plus (KGP) estimate. The KGP estimate is used to predict blood characteristics, such as hemoglobin concentration and hematocrit. The KGP is estimated in three distinct phases. First, the images are screened to measure a mean image intensity and motion blur. Second, each image is analyzed to identify background curvate, and create vessel, background and diameter masks. To identify background curvate, the images are analyzed to detect shadows caused by larger blood vessels in the background image. The diameter and area of the vessels are also calculated. During the Prediction and Calibration phase, the images are screened to eliminate all images failing thresholds for mean intensity, motion blur, background curvature and area-to-perimeter ratio. Finally, the KGP estimate is determined from the selected images.

### BACKGROUND OF THE INVENTION

1. Field of the Invention

The present invention relates generally to reflected light analysis. More particularly, the invention relates to the use of reflected spectral imaging to analyze visualizable components of a fluid flowing in a tubular system. Still more particularly, the invention relates to the use of reflected spectral imaging to analyze the components of blood in a mammalian, especially human, vascular system.

2. Related Art

Widely accepted medical school doctrine teaches that the complete blood count including the white blood cell differential (CBC+Diff) is one of the best tests to assess a patient's overall health. With it, a physician can detect or diagnose anemia, infection, blood loss, acute and chronic diseases, allergies, and other conditions. CBC+Diff analyses provide comprehensive information on constituents in blood, including the number of red cells, the hematocrit, the hemoglobin concentration, and indices that portray the size, shape, and oxygen-carrying characteristics of the entire red blood cell (RBC) population. The CBC+Diff also includes the number and types of white blood cells and the number of platelets. The CBC+Diff is one of the most frequently requested diagnostic tests with about two billion done in the United States per year.

A conventional CBC+Diff test is done in an “invasive” manner in which a sample of venous blood is drawn from a patient through a needle, and submitted to a laboratory for analysis. For example, a phlebotomist (an individual specially trained in drawing blood) collects a sample of venous blood into a tube containing an anticoagulant to prevent the blood from clotting. The sample is then sent to a hematology laboratory to be processed, typically on automated, multiparameter analytical instruments, such as those manufactured by Coulter Diagnostics of Miami, Fla. The CBC+Diff test results are returned to the requesting physician, typically on the next day.

In medical diagnosis it is often necessary to measure other types of blood components, such as non-cellular constituents present in the plasma component of blood. Such constituents can include, for example, blood gases and bilirubin. Bilirubin is a reddish to yellow pigment produced in the metabolic breakdown of hemoglobin and other proteins. Bilirubin is removed from the blood by the liver and is excreted from the body. However, the livers of newborn children, especially premature babies, cannot process bilirubin effectively.

The birth process often results in extensive bruising, resulting in blood escaping into the tissues where it is broken down metabolically. For this and other medical causes, bilirubin may accumulate in the blood stream. If bilirubin levels rise high enough, it begins to be deposited in other body tissues causing jaundice. Its first appearance is in the eye. At still higher levels, deposition begins in deeper tissues, including the brain, and can result in permanent brain damage.

The most common method for bilirubin analysis is through an in vitro process. In such an in vitro process, a blood sample is invasively drawn from the patient. The formed elements (red blood cells and other cells) are separated by centrifugation and the remaining fluid is reacted chemically and analyzed spectrophotometrically.

Invasive techniques, such as for conventional CBC+Diff tests and bilirubin analysis, pose particular problems for newborns because their circulatory system is not yet fully developed. Blood is typically drawn using a “heel stick” procedure wherein one or more punctures are made in the heel of the newborn, and blood is repeatedly squeezed out into a collecting tube. This procedure is traumatic even for an infant in good health. More importantly, this procedure poses the risk of having to do a blood transfusion because of the low total blood volume of the infant. The total blood volume of the newborn infant is 60-70 cc/kg body weight. Thus, the total blood volume of low birth weight infants (under 2500 grams) cared for in newborn intensive care units ranges from 45-175 cc. Because of their low blood volume and delay in production of red cells after birth, blood sampling from preterm infants and other sick infants frequently necessitates transfusions for these infants. Blood bank use for transfusion of infants in neonatal intensive care units is second only to the usage for cardiothoracic surgery. In addition to newborns, invasive techniques are also particularly stressful for, and/or difficult to carryout on, children, elderly patients, bum patients, and patients in special care units.

A hierarchical relationship exists between the laboratory findings and those obtained at the physical examination. The demarcation between the physical findings of the patient and the laboratory findings are, in general, the result of technical limitations. For instance, in the diagnosis of anemia (defined as low hemoglobin concentration), it is frequently necessary to quantify the hemoglobin concentration or the hematocrit in order to verify the observation of pallor. Pallor is the lack of the pink color of skin which frequently signals the absence or reduced concentration of the heavily red pigmented hemoglobin. However, there are some instances in which pallor may result from other causes, such as constriction of peripheral vessels, or being hidden by skin pigmentation. Because certain parts of the integument are less affected by these factors, clinicians have found that the pallor associated with anemia can more accurately be detected in the mucous membrane of the mouth, the conjunctivae, the lips, and the nail beds. A device which is able to rapidly and non-invasively quantitatively determine the hemoglobin concentration directly from an examination of one or more of the foregoing areas would eliminate the need to draw a venous blood sample to ascertain anemia. Such a device would also eliminate the delay in waiting for the laboratory results in the evaluation of the patient. Such a device also has the advantage of added patient comfort.

Soft tissue, such as mucosal membranes or unpigmented skin, do not absorb light in the visible and near-infrared, i.e., they do not absorb light in the spectral region where hemoglobin absorbs light. This allows the vascularization to be differentiated by spectral absorption from surrounding soft tissue background. However, the surface of soft tissue strongly reflects light and the soft tissue itself effectively scatters light after penetration of only 100 microns. Therefore, in vivo visualization of the circulation is difficult because of poor resolution, and generally impractical because of the complexities involved in compensating for multiple scattering and for specular reflection from the surface. Studies on the visualization of cells in the microcirculation consequently have been almost exclusively invasive, using a thin section (less than the distance for multiple scattering) of tissue containing the microcirculation, such as the mesentery, that can be observed by a microscope using light transmitted through the tissue section. Other studies have experimented with producing images of tissues from within the multiple scattering region by time gating (see, Yodh, A. and B. Chance, Physics Today, March, 1995, 34-40). However, the resolution of such images is limited because of the scattering of light, and the computations for the scattering factor are complex.

Spectrophotometry involves analysis based on the absorption or attenuation of electromagnetic radiation by matter at one or more wavelengths of light. The instruments used in this analysis are referred to as spectrophotometers. A simple spectrophotometer includes: a source of radiation, such as, e.g., a light bulb; a spectral selection means, such as a monochromator containing a prism or grating or colored filter; and one or more detectors, such as, e.g., photocells, which measure the amount of light transmitted and/or reflected by the sample in the selected spectral region.

In opaque samples, such as solids or highly absorbing solutions, the radiation reflected from the surface of the sample maybe measured and compared with the radiation reflected from a non-absorbing or white sample. If this reflectance intensity is plotted as a function of wavelength, it gives a reflectance spectrum: Reflectance spectra are commonly used in matching colors of dyed fabrics or painted surfaces. However, because of its limited range and inaccuracy, reflection spectrophotometry has been used primarily in qualitative rather than quantitative analysis. On the other hand, transmission spectrophotometry is conventionally used for quantitative analysis because Beer's law (inversely relating the logarithm of measured intensity linearly to concentration) can be used.

Reflective spectrophotometry is conventionally avoided for quantitative analysis because specularly reflected light from a surface limits the available contrast (black to white or signal to noise ratio), and, consequently, the measurement range and linearity. Because of surface effects, measurements are usually made at an angle to the surface. However, only for the special case of a Lambertian surface will the reflected intensity be independent of the angle of viewing. Light reflected from a Lambertian surface appears equally bright in all directions (cosine law). However, good Lambertian surfaces are difficult to obtain. Conventional reflection spectrophotometry presents an even more complicated relationship between reflected light intensity and concentration than exists for transmission spectrophotometry which follows Beer's law. Under the Kubelka-Munk theory applicable in reflection spectrophotometry, the intensity of reflected light can be related indirectly to concentration through the ratio of absorption to scattering.

Some imaging studies have been done in the reflected light of the microcirculation of the nail beds on patients with Raynauds, diabetes, and sickle cell disease. These studies were done to obtain experimental data regarding capillary density, capillary shape, and blood flow velocity, and were limited to gross physical measurements on capillaries. No spectral measurements, or individual cellular measurements, were made, and Doppler techniques were used to assess velocity. The non-invasive procedure employed in these studies could be applied to most patients, and in a comfortable manner.

One non-invasive device for iii vivo analysis is disclosed in U.S. Pat. No. 4,998,533 to Winkei-nan. The Winkelman device uses image analysis and reflectance spectrophotometry to measure individual cell parameters such as cell size. Measurements are taken only within small vessels, such as capillaries where individual cells can be visualized. Because the Winkelman device takes measurements only in capillaries, measurements made by the Winkelman device will not accurately reflect measurements for larger vessels. This inaccuracy results from the constantly changing relationship of volume of cells to volume of blood in small capillaries resulting from the non-Newtonian viscosity characteristic of blood. Consequently, the Winkelman device is not capable of measuring the central or true hematocrit, or the total hemoglobin concentration, which depend upon the ratio of the volume of red blood cells to that of the whole blood in a large vessel such as a vein.

The Winkelman device measures the number of white blood cells relative to the number of red blood cells by counting individual cells as they flow through a micro-capillary. The Winkelman device depends upon accumulating a statistically reliable number of white blood cells in order to estimate the concentration. However, blood flowing through a micro-capillary will contain approximately 1000 red cells for every white cell, making this an impractical method. The Winkelman device does not provide any means by which platelets can be visualized and counted. Further, the Winkelman device does not provide any means by which the capillary plasma can be visualized, or the constituents of the capillary plasma quantified. The Winkelman device also does not provide a means by which abnormal constituents of blood, such as tumor cells, can be detected.

Another non-invasive device for in vivo analysis is disclosed in commonly assigned U.S. Pat. No. 5,983,120, issued Nov. 9, 1999, in the names of Warren Groner and Richard G. Nadeau, and entitled “Method and Apparatus for Reflected Imaging Analysis” (hereinafter referred to as “the '120 patent”), or in commonly assigned U.S. Pat. No. 6,104,939, issued Aug. 15, 2000, in the names of Warren Groner and Richard G. Nadeau, and entitled “Method and Apparatus for Reflected Imaging Analysis” (hereinafter referred to as “the '939 patent”). The disclosure of the '120 patent and the '939 patent are incorporated herein by reference as though set forth in its entirety. The device of the '120 patent or the '939 patent provides for complete non-invasive in vivo analysis of a vascular system. This device provides for high resolution visualization of blood cell components (red blood cells, white blood cells, and platelets), blood rheology, blood vessels, and vascularization throughout the vascular system. The device of the '120 patent or the '939 patent allows quantitative determinations to be made for blood cells, normal and abnormal contents of blood cells, as well as for normal and abnormal constituents of blood plasma.

The device of the '120 patent or the '939 patent captures a raw reflected image of a blood sample, and normalizes the image with respect to the background to form a corrected reflected image. An analysis image is segmented from the corrected reflected image to include a scene of interest for analysis. The method and apparatus disclosed in the '120 patent or the '939 patent can be used to determine such characteristics as the hemoglobin concentration per unit volume of blood, the number of white blood cells per unit volume of blood, a mean cell volume, the number of platelets per unit volume of blood, and the hematocrit.

To accurately determine the blood characteristics, however, the images need to be screened to identify images having good measurable properties. The measurements taken from the images also need to be screened, normalized and corrected to obtain better estimates of the true value of the blood characteristics.

Thus, there is a need in the art for a method and device that selects images having good measurable properties and provides reliable, quantitative estimates of blood cells, normal and abnormal contents of blood cells, and normal and abnormal constituents of blood plasma by using non-invasive in vivo analysis.

### SUMMARY OF THE INVENTION

The present invention is directed to processing reflected spectral images of a microcirculatory system to measure the volume and concentration of a blood vessel, including arteries, veins and capillaries. Basically, the method and apparatus of the present invention analyze the spectral image (also referred to herein as “blood sample” or “image”) to identify vessel structure and measure the light absorption in the vessel to develop what is referred to as a contrast gradient plus (KGP) estimate. The KGP estimate is used to measure blood characteristics, such as the hemoglobin concentration and hematocrit of the blood sample.

The KGP estimate is calculated in three steps or phases: Screening, Analysis, and Calibration & Prediction. The images are first screened to measure mean image intensity and motion blur parameters. It should be noted that, in an embodiment, the screening phase is used only to detect certain parameters. No image is eliminated at this step in the process. However, in another embodiment, an optimal screening threshold can be implemented to reject images that do not meet the screening threshold at this step.

Next, the spectral image is analyzed to identify background curvature, and create vessel, background and diameter masks. First, to identify background curvature, the images are analyzed to identify shadows caused by larger blood vessels in the background of the smaller blood vessel(s) being evaluated. Afterwards, the image is processed to segment all vascular structure from nonvascular regions. During this process, a model of the vascular structure is developed to create a vessel image. A model also is developed to create a background image. The diameter and area of the vessels are calculated, as well. It should be noted that these steps may occur concurrently or in a different order.

Finally, the images enter a Prediction and Calibration phase where the images are subsequently screened to eliminate all images that fail to pass certain thresholds for motion blur and background curvature criteria. Of the remaining images, the vessel area is used to detect anemia and select better images for anemic patients. At last, the KGP estimate is determined from the selected images.

The method of the present invention can be used to determine various characteristics of blood. Such characteristics can include the hemoglobin concentration per unit volume of blood, the number of white blood cells per unit volume of blood, a mean cell volume, a mean cell hemoglobin concentration, the number of platelets per unit volume of blood, and the hematocrit.

The method is used to perform inZ vivo analysis of blood in large vessels, and in vivo analysis of blood in small vessels to determine blood parameters such as concentrations and blood cell counts. The method of the present invention can also be used to conduct non-invasive in vivo analysis of non-cellular characteristics of capillary plasma. The method of the present invention can also be used to perform in vitro analyses by imaging blood in, for example, a tube or flow cell.

The method of the present invention can also be used to analyze other types of fluids containing visualizable components. The reflected spectral imaging system can be used to analyze fluids for particulate impurities. It is only necessary that the walls of the fluid path be sufficiently transparent to permit light to pass through the walls of the fluid path to image the fluid and any impurities flowing in the path.

Features and Advantages

It is a feature of the present invention that it provides for non-invasive in vivo analysis of the vascular system.

It is a further feature of the present invention that quantitative analyses of both formed blood components (red blood cells, white blood cells, and platelets) and non-formed blood components, such as capillary plasma, can be done.

It is yet a further feature of the present invention that per unit volume or concentration measurements, such as hemoglobin, hematocrit, and blood cell counts, can be made through the use of reflected spectral images of the vascular system.

It is yet a further feature of the present invention that blood cells, blood vessels, and capillary plasma can be visualized and segmented into an analysis image.

A still farther feature of the present invention is that it can be used to determine characteristics, such as the hemoglobin concentration per unit volume of blood, the number of white blood cells per unit volume of blood, the mean cell volume, the mean cell hemoglobin concentration, the number of platelets per unit volume of blood, and the hematocrit through the use of reflected spectral imaging.

An advantage of the present invention is that it provides a means for the rapid, non-invasive measurement of clinically significant parameters of the CBC+Diff test. It advantageously provides immediate results. As such, it can be used for point-of-care testing and diagnosis.

A further advantage of the present invention is that it eliminates the invasive technique of drawing blood. This eliminates the pain and difficulty of drawing blood from newborns, children, elderly patients, bum patients, and patients in special care units. The present invention is also advantageous in that it obviates the risk of exposure to AIDS, hepatitis, and other blood-borne diseases.

A still further advantage of the present invention is that it provides for overall cost savings by eliminating sample transportation, handling, and disposal costs associated with conventional invasive techniques.

A still further advantage of the present invention is that it provides for substantially improved range and accuracy for reflection spectrophotometry. The present invention is also advantageous in that it permits use of a simple relationship between concentration and intensity.

A still further advantage of the present invention is that it provides for improved visualization of reflected images of any object, and for quantitative and qualitative analyses of these reflected images.

### BRIEF DESCRIPTION OF THE FIGURES

The present invention is described with reference to the accompanying drawings. In the drawings, like reference numbers indicate identical or functionally similar elements. Additionally, the left-most digit(s) of a reference number identifies the drawing in which the reference number first appears.

FIG. 1 shows a flow chart representing the general operational flow according to an embodiment of a method of the present invention;

FIG. 2 shows a flow chart illustrating step 150 shown in FIG. 1;

FIG. 3 shows a flow chart illustrating step 215 shown in FIG. 2;

FIG. 4 shows a flow chart representing the general operational flow according to an embodiment of the motion detection method of the present invention;

FIG. 5A shows a flow chart representing the general operational flow according to an embodiment for calculating parameters and images required for the diameter estimation method of the present invention;

FIG. 5B shows a flow chart representing the general operational flow according to an embodiment of the diameter estimation method of the present invention;

FIG. 6 shows a flow chart illustrating step 210 shown in FIG. 2;

FIG. 7 shows a flow chart representing the general operational flow according to an embodiment of the calibration and prediction method of the present invention;

FIG. 8 is a block diagram of an example computer system useful for implementing the present invention; and

FIG. 9 shows a flow chart representing the general operational flow according to an embodiment of the area-to-perimeter estimation method of the present invention.

### DETAILED DESCRIPTIOIZ OF THE PREFERRED EMBODINIENTS

I. Overview of the Present Invention

The present invention is directed to a method and apparatus for analysis, particularly non-invasive, in vivo analysis of a subject's vascular system. The in vivo measurements discussed herein can also be performed in vitro by imaging blood in, for example, a tube or flow cell, as would be apparent to a person skilled in the relevant art(s). The in vivo method is carried out by imaging a portion of the subject's vascular system. For example, the image can be created from a sub-surface region of a subject's tissues or organs. The tissue covering the imaged portion must be traversed by light without multiple scattering to obtain a reflected image. In order to form an image, two criteria must be met. First, there must be image contrast resulting from a difference in the optical properties, such as absorption, index of refraction, or scattering characteristics, between the subject to be imaged and its surroundings or background. Second, the light that is collected from the subject must reach an image capturing means without substantial scattering, i.e., the reflected image must be captured from a depth that is less than the multiple scattering length. As used herein, “image” refers to any image that satisfies the foregoing two criteria. As used herein, “reflected image” refers to the image of a subject in reflected light. The resolution required for capturing the image is dictated by the spatial homogeneity of the imaged portion. For example, a reflected image of individual cells requires high resolution. A reflected image of large vessels can be done with low resolution. A reflected image suitable for making a determination based on pallor requires very low resolution.

The tissue covering the imaged portion is thus preferably transparent to light, and relatively thin, such as the mucosal membrane on the inside of the lip of a human subject. As used herein, “light” refers generally to electromagnetic radiation of any wavelength, including the infrared, visible, and ultraviolet portions of the spectrum. A particularly preferred portion of the spectrum is that portion where there is relative transparency of tissue, such as in the visible and near-infrared wavelengths. It is to be understood that for the present invention, light can be coherent light or incoherent light, and illumination may be steady or in pulses of light.

The reflected image is corrected to form a corrected reflected image. The correction to the reflected image is done, for example, to isolate particular wavelengths of interest, or to extract a moving portion of the image from a stationary portion of the image. A scene is segmented from the corrected reflected image to form an analysis image. The analysis image is then analyzed for the desired characteristic of the subject's vascular system.

The method of the present invention can be used for analysis in large and small vessels, including capillary plasma. As used herein, “large vessel” refers to a vessel in the vascular system of sufficient size so that a plurality of red blood cells flow side-by-side through it. “Small vessel” refers to a vessel in the vascular system of a size so that red blood cells flow substantially “single file” through it. As explained in more detail below, the present invention uses reflectance, not transmission, for the images that are analyzed. That is, the image is made by “looking at” the vascular system, rather than by “looking through” the vascular system. However, because of the features of the imaging system used in the present invention, as described in detail in the above-referenced '120 patent or the '939 patent, the image appears to be of the transmission type. For this reason, Beer's law can be applied to quantitatively measure the images. Per unit volume or concentration measurements can be made directly from the images. Therefore, although the present invention uses reflectance, it would be apparent to a person skilled in the relevant art(s) that the method of the present invention can be used on both transmitted and reflected images.

By using the method of the present invention to provide a reflected spectral image of large vessels, the hemoglobin (Hb), hematocrit (Hct), and white blood cell count (WBC) parameters can be directly determined. By using the method of the present invention to provide a reflected spectral image of small vessels, mean cell volume (MCV), mean cell hemoglobin concentration (MCHC), and platelet count (Plt) can be directly determined.

Human blood is made up of formed elements and plasma. There are three basic types of formed blood cell components: red blood cells (erythrocytes); white blood cells (leukocytes); and platelets. As noted above, red blood cells contain hemoglobin that carries oxygen from the lungs to the tissues of the body. White blood cells are of approximately the same size as red blood cells, but do not contain hemoglobin. A normal healthy individual will have approximately 5,000,000 red blood cells per cubic millimeter of blood, and approximately 7,500 white blood cells per cubic millimeter of blood. Therefore, a normal healthy individual will have approximately one white blood cell for every 670 red blood cells circulating in the vascular system.

A complete blood count (CBC) without white blood cell differential measures eight parameters: (1) hemoglobin (Hb); (2) hematocrit (Hct); (3) red blood cell count (RBC); (4) mean cell volume (MCV); (5) mean cell hemoglobin (MCH); (6) mean cell hemoglobin concentration (MCHC); (7) white blood cell count (WBC); and (8) platelet count (Plt). The first six parameters are referred to herein as RBC parameters. Concentration measurements (measurements per unit volume of blood) are necessary for producing values for Hb, Hct, RBC, WBC, and Plt. Hb is the hemoglobin concentration per unit volume of blood. Hct is the volume of cells per unit volume of blood. Hct can be expressed as a percentage, i.e.,:

(cell volume÷volume of blood)×100% (Eqn. 1)

RBC is the number of red blood cells per unit volume of blood. WBC is the number of white blood cells per unit volume of blood. Plt is the number of platelets per unit volume of blood.

Red cell indices (MCV, MCH, and MCHC) are cellular parameters that depict the volume, hemoglobin content, and hemoglobin concentration, respectively, of the average red cell. The red cell indices may be determined by making measurements on individual cells, and averaging the individual cell measurements. Red cells do not change volume or lose hemoglobin as they move through the vascular system. Therefore, red cell indices are constant throughout the circulation, and can be reliably measured in small vessels. The three red cell indices are related by the equation:

MCHC=MCH÷MCV (Eqn. 2)

Thus, only two red cell indices are independent variables.

To determine values for the six RBC parameters listed above, the following two criteria must be met. First, three of the parameters must be independently measured or determined. That is, three of the parameters must be measured or determined without reference to any of the other of the six parameters. Second, at least one of the three independently measured or determined parameters must be a concentration parameter (per unit volume of blood). Therefore, values for the six key parameters can be determined by making three independent measurements, at least one of which is a concentration measurement which cannot be made in a small vessel.

As disclosed in the '120 patent or the '939 patent, Hb and Hct can be directly measured by reflected spectral imaging of large vessels, and MCV can be directly measured by reflected spectral imaging of small vessels. In this manner, three parameters are independently measured, and two of the parameters (Hb and Hct) are concentration parameters measured per unit volume of blood. As such, the six RBC parameters listed above can be determined in the following manner:

| | |
|---|---|
| Hb | Directly measured |
| Hct | Directly measured |
| RBC | Hct ÷ MCV |
| MCV | Directly measured |
| NCH | MCV × (Hb ÷ Hct) |
| MCHC | Hb ÷ Hct |

Also, as disclosed in the '120 patent or the '939 patent, Hb can be directly measured by reflected spectral imaging of large vessels, and MCV and MCHC can be directly measured by reflected spectral imaging of small vessels. In this manner, three parameters are independently measured, and one of the parameters (Hb) is a concentration parameter measured per unit volume of blood. As such, the six RBC parameters listed above can be determined in the following manner:

| | |
|---|---|
| Hb | Directly measured |
| Hct | Hb ÷ NCHC |
| RBC | Hb ÷ (MCV × MCHC) |
| MCV | Directly measured |
| MCH | MCV × MCHC |
| MCHC | Directly measured |

Concentration measurements are measurements per unit volume. As discussed above, a measurement made per unit area is proportional to a measurement made per unit volume (volume measurement with constant depth) when the depth of penetration is constant. The depth of penetration is a function of wavelength, the size of the particles with which it interacts, and refractive index. For blood, the particle size and index of refraction are essentially constant. Consequently, the depth of penetration will be constant for a particular wavelength.

Hemoglobin is the main component of red blood cells. Hemoglobin is a protein that serves as a vehicle for the transportation of oxygen and carbon dioxide throughout the vascular system. Hemoglobin absorbs light at particular absorbing wavelengths, such as 550 nm, and does not absorb light at other non-absorbing wavelengths, such as 650 nm. Under Beer's law, the negative logarithm of the measured transmitted light intensity is linearly related to concentration. As explained more fully in the '120 patent or the '939 patent, a spectral imaging apparatus can be configured so that reflected light intensity follows Beer's law. Assuming Beer's law applies, the concentration of hemoglobin in a particular sample of blood is linearly related to the negative logarithm of light reflected by the hemoglobin. The more 550 nm light absorbed by a blood sample, the lower the reflected light intensity at 550 nm, and the higher the concentration of hemoglobin in that blood sample. The concentration of hemoglobin can be computed by taking the negative logarithm of the measured reflected light intensity at an absorbing wavelength such as 550 nm. Therefore, if the reflected light intensity from a particular sample of blood is measured, the concentration in the blood of such components as hemoglobin can be directly determined.

The method of the present invention can also be used to determine the hematocrit (Hct). The difference between hemoglobin (which is the grams of hemoglobin per volume of blood) and hematocrit (which is the volume of blood cells per volume of blood) is determined by the concentration of hemoglobin within the cells which determines the index of refraction of the cells. Hence, measurements in which the image contrast between the circulation and the background is achieved principally by the scattering properties of the circulation will be related to the hematocrit and those obtained principally by the absorbing properties will be related primarily to the hemoglobin. For example, the microvascular system beneath the mucosal membrane on the inside of the lip of a human subject can be imaged to produce a raw reflected image whose contrast is determined by a difference in the scattering properties of the blood cells.

As disclosed in the '120 patent or the '939 patent, a spectral imaging apparatus includes a light source that is used to illuminate the portion of the subject's vascular system to be imaged. The reflected light is captured by an image capturing means. Suitable image capturing means include, but are not limited to, a camera, a film medium, a photocell, a photodiode, or a charge coupled device camera. An image correcting and analyzing means, such as a computer, is coupled to the image capturing means for carrying out image correction, scene segmentation, and blood characteristic analysis.

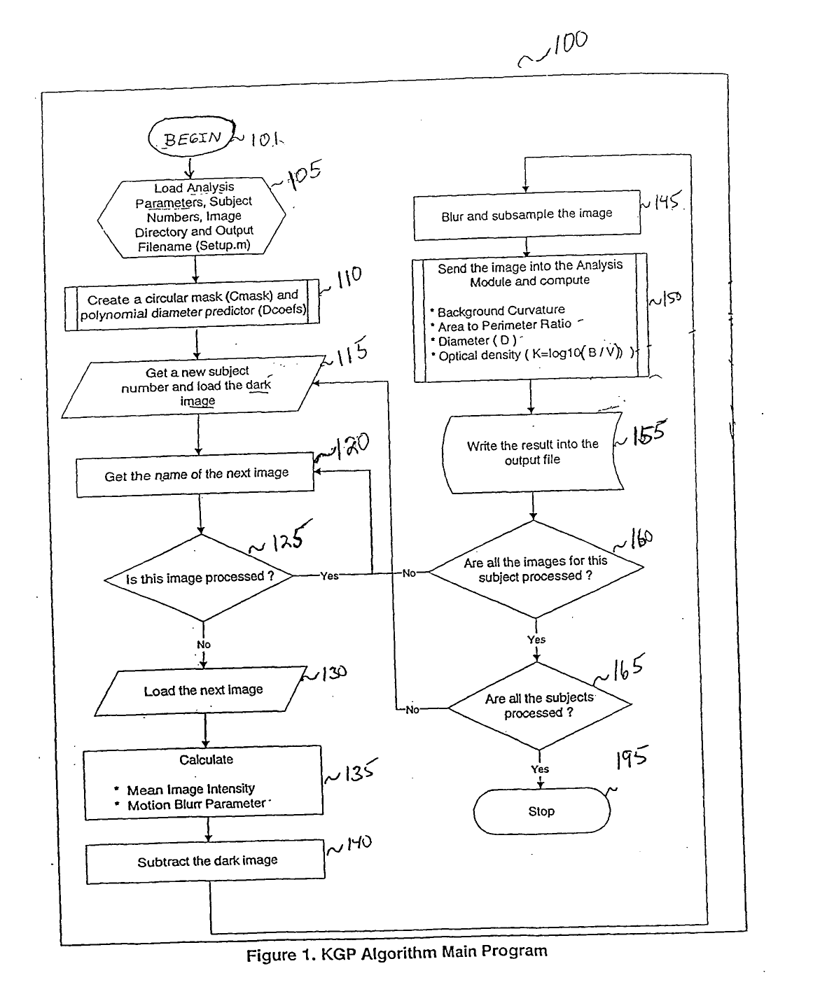

To implement the method of the present invention, the reflected image is captured and processed to select images having good measurable property. The selected image(s) is screened and analyzed to produce a reliable measurement of the concentration and volume of blood characteristics, such as hematocrit or hemoglobin. FIG. 1 illustrates a general operational flow of one embodiment of the present invention. More specifically, flowchart 100 shows an example of a process for analyzing a spectral image of a blood or tissue sample. The spectral image can be obtained from a spectral imaging apparatus preferably, but not necessarily, of the type described in the '120 patent or the '939 patent. Nonetheless, the spectral image can be obtained from any type of imaging apparatus designed for tissue or blood analysis, as would be apparent to a person skilled in the relevant art(s).

FIG. 1 starts at step 101 and passes immediately to step 105, where the spectral images are retrieved from a memory source or image directory. The images can be retrieved from an input file stored in a temporary or permanent memory location on a hard disk drive or removable storage device, such as a floppy diskette, magnetic tape, optical disks, etc., or the like, as would be apparent to a person skilled in the relevant art(s). The input file also includes the subject number or other data used to identify the patient. At step 105, an output file is also created to store relevant test results, as described below in further detail. Also, at step 105, the requisite load analysis parameters are downloaded for use during subsequent calculations. The load analysis parameters include various threshold values used to analyze or screen the Laplacian masks, linear filtering, motion blur, and the like.

The KGP is estimated in three distinct phases: a screening phase, analysis phase, and prediction and calibration phase. The Screening Phase and Analysis Phase are illustrated in FIG. 1. Basically, the Screening Phase commences at step 135 after the image has been selected at step 130. During the Screening Phase, the image is processed to calculate a mean image intensity and motion blur parameter. The image enters the Analysis Phase at step 150 to measure certain parameters used to calculate the hemoglobin and hematocrit, namely background curvature, area-to-perimeter ratio, vessel diameter and optical density. The test results are written to the output file at step 155 and the process is repeated for the next image at step 160.

II. Screening Phase

The Screening Phase is used to evaluate the quality of the spectral images by calculating two parameters: mean image intensity and motion blur. These parameters are used to select images whose parameters are within certain thresholds. These images will provide the better samples for measuring the blood characteristics. The mean image intensity represents the average of the intensity of all pixels in the spectral image. The mean image intensity is compared to a threshold value to determine if the image is too dark or too bright for analysis.

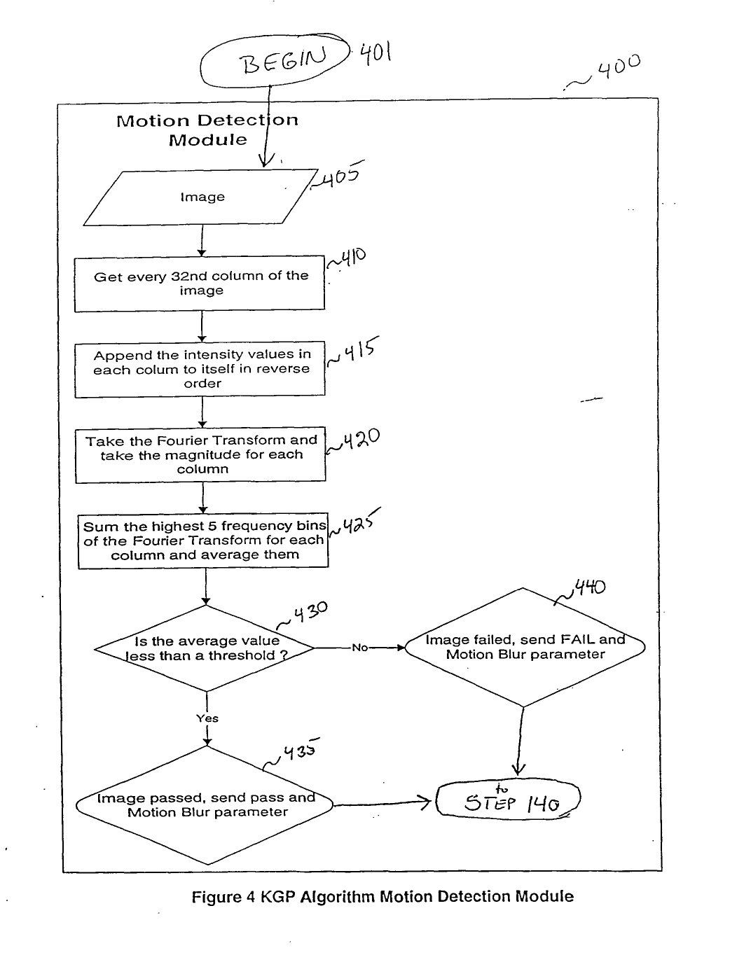

To calculate the motion blur parameters, the method of the present invention uses a Fourier Transform to estimate the amount of inter-field motion in the images as an estimate of intra-field blur. The images with considerable motion blur exhibit higher energy in the high frequency of the Fourier Transform. One embodiment of the general operational flow for detecting motion blur is illustrated as control flow 400 in FIG. 4. For example, at step 410, every thirty-second column of the image is sampled. At step 415, the intensity values in each column are appended in reverse order. At step 420, a Fourier Transform is used to calculate the magnitude for each column. Step 425 sums and averages the five highest frequency bins of the Fourier Transform for each column. If the average value is equal to or exceeds a specific motion blur threshold, the image is deemed to have failed and the average value for the image is recorded as the image's motion blur parameter at step 440. If the average value does not exceed the motion blur threshold, the image is deemed to have passed and the average value is recorded as its motion blur parameter at step 435. Referring to FIG. 1, this binary result, i.e. motion blur parameter, is calculated at step 135, and then control flow 400 proceeds to step 140. As described, FIG. 4 represents only an exemplary embodiment for detecting motion blur. As would be apparent to one skilled in the relevant art(s), other methodologies for detecting the presence of significant energy in the high frequency region can be implemented and are considered to be within the scope of the present invention.

The motion blur threshold is one of the several load analysis parameters selected at Step 105 in FIG. 1. The motion blur threshold is based on various factors, such as the type of spectral imaging apparatus. For example, the motion blur threshold can be one of several parameters used to calibrate the spectral imaging apparatus to improve the accuracy of the measurements and calculations resulting from the measurements.

After the screening parameters have been calculated to measure the quality of the image, a dark image is subtracted from the spectral image. At step 140, the dark image is used as a zero offset to normalize the spectral image. Then, the method of the present invention analyzes the normalized spectral image to evaluate the content of the image. In one embodiment, all images are analyzed regardless of the screening results. The rejection of measurements taken from poor images is implemented at the Calibration and Prediction Phase. In another embodiment, optimal screening thresholds are entered at step 105. The images that do not meet the screening thresholds are rejected and are not analyzed during the Analysis Phase.

III. Analysis Phase

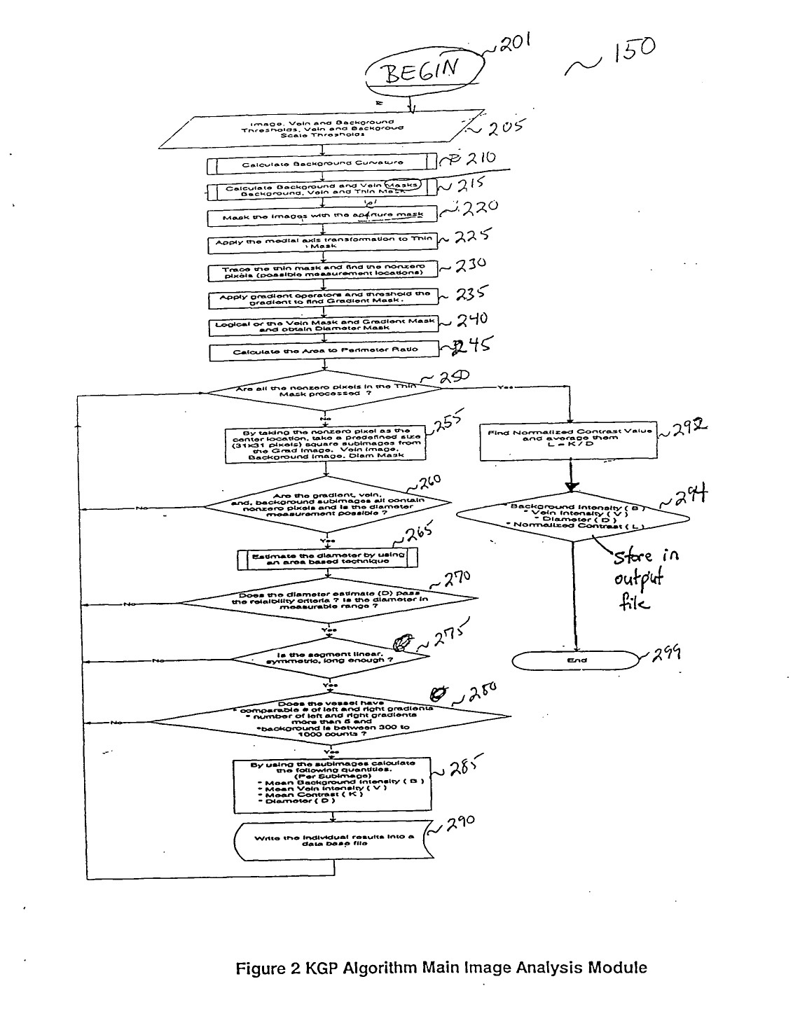

During the Analysis Phase, three attributes are evaluated: background curvature, vascular structure and geometric properties. FIG. 2 illustrates an operational flow of one embodiment of the Analysis Phase. More specifically, FIG. 2 is one embodiment of a more detailed description of process step 150 from FIG. 1. The background curvature is calculated at step 210. The background curvature is used to identify and measure the effects of larger background blood vessels that cast shadows and compromise the details of the smaller vessels located in the foreground. This shadowing effect causes “curvature” in the image when it is observed in the intensity domain as a three dimensional map. This shadowing or curvature interferes with the reliability of the optical density measurements in the smaller vessels. In other words, the background curvature can attribute to an underestimate of the optical density which produces an underestimate of the hemoglobin of the subject. Therefore, this information can be used to eliminate images having background curvature that exceeds a specific threshold, as discussed below.

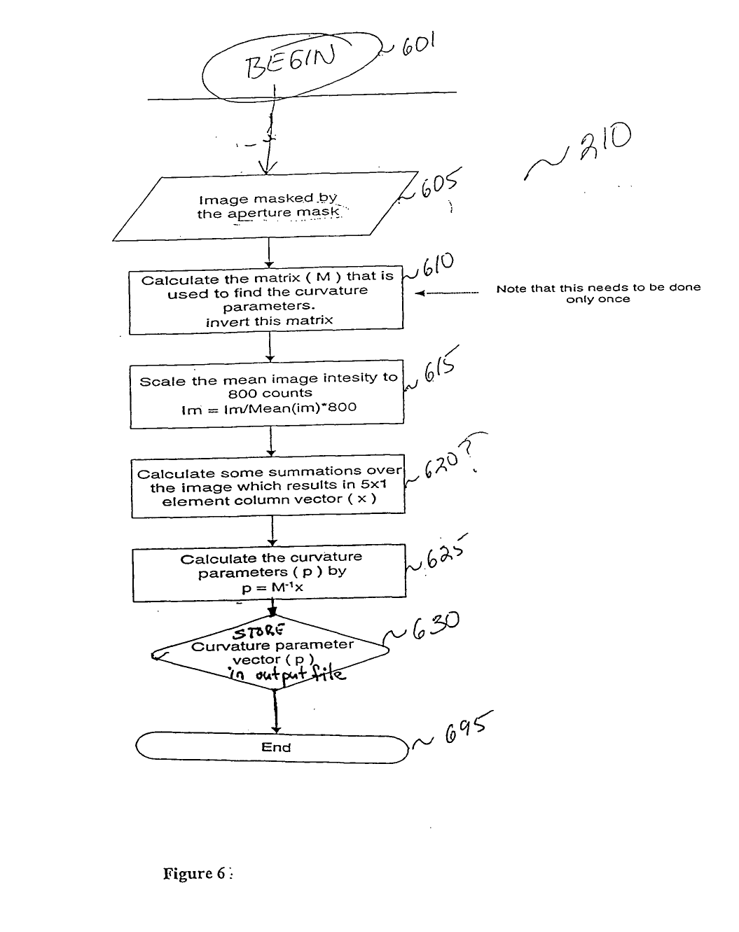

A more detailed illustration of one embodiment of the process for calculating the background curvature is shown in FIG. 6. More particularly, FIG. 6 illustrates one embodiment of step 210. Background curvature can be estimated by fitting a second order three dimensional polynomial surface model to the spectral image. The model is given by:

F(x,y)=a₀+a₁x+a₂y+a₃x²+a₄y²+a₅xy (Eqn. 3)

where x and y are the row and column coordinates of the spectral image, and a₀, a₁, a₂, a₃, a₄ and a₅ are the constant parameters of the model. The parameters a, a₁, a₂, a₃, a₄ and a₅ can be found by using a least squares minimization between the image and the background curvature model, such as:

Min(Σ_{x,y}(Im(x,y)−(a₀+a₁x+a₂y+a₃x²+a₄y²+a₅x y))²) (Eqn. 4)

The solution of the minimization can be obtained by first taking the derivative of the above equation and setting them to zero and to create the following matrix:

$$[ \begin{matrix}{\sum }_{xy}^{} 1 & {\sum }^{} x & {\sum }^{} y & {\sum }^{} {x}^{2} & {\sum }^{} {y}^{2} & {\sum }^{} xy \\ {\sum }^{} x & {\sum }^{} {x}^{2} & {\sum }^{} xy & {\sum }^{} {x}^{3} & {\sum }^{} {xy}^{2} & {\sum }^{} {x}^{2} y \\ {\sum }^{} y & {\sum }^{} xy & {\sum }^{} {y}^{2} & {\sum }^{} {x}^{2} y & {\sum }^{} {y}^{3} & {\sum }^{} {xy}^{2} \\ {\sum }^{} {x}^{2} & {\sum }^{} {x}^{3} & {\sum }^{} {x}^{2} y & {\sum }^{} {x}^{4} & {\sum }^{} {x}^{2} {y}^{2} & {\sum }^{} {x}^{3} y \\ {\sum }^{} {y}^{2} & {\sum }^{} {xy}^{2} & {\sum }^{} {y}^{2} & {\sum }^{} {x}^{2} {y}^{2} & {\sum }^{} {y}^{4} & {\sum }^{} {xy}^{3} \\ {\sum }^{} xy & {\sum }^{} {x}^{2} y & {\sum }^{} {xy}^{2} & {\sum }^{} {x}^{3} y & {\sum }^{} {xy}^{3} & {\sum }^{} {x}^{2} {y}^{2}\end{matrix} ] [ \begin{matrix}{a}_{0} \\ {a}_{1} \\ {a}_{2} \\ {a}_{3} \\ {a}_{4} \\ {a}_{5}\end{matrix} ] = [ \begin{matrix}{\sum }_{xy}^{} F \\ {\sum }_{xy}^{} xF \\ {\sum }_{}^{} yF \\ {\sum }^{} {x}^{2} F \\ {\sum }^{} {y}^{2} F \\ {\sum }^{} xyF\end{matrix} ] \qquad ( Eqn . 5 )$$

The above matrix of equations can be solved for a₀, a₁, a₂, a₃, a₄ and a₅ to obtain the model's parameters. The parameter a₀ gives the overall constant or uniform average level of the image. The parameters a₁, a₂ and a₅ are a measure of the tilt of the background. The parameters a₃ and a₄ are a measure of the curvature of the image. Parameters a₃ and a₄ are used to measure curvature of the background and screen out the images that have high background curvature. For example, the following function:

Max {a₃, a₄} (Eqn. 6)

can be used as a thresholding criterion to screen out images with high background curvature. Other functional combinations of the parameters a₀, a₁, a₂, a₃, a₄ and a₅ can be used to evaluate other properties of the spectral image.

Referring to FIG. 6, matrix equation 5 is calculated at step 610 and solved at steps 615-630 to determine the background curvature parameters a₀, a₁, a₂, a₃, a₄ and a₅ (also shown as p in FIG. 6). The parameters are calculated for each image and compared to the background threshold.

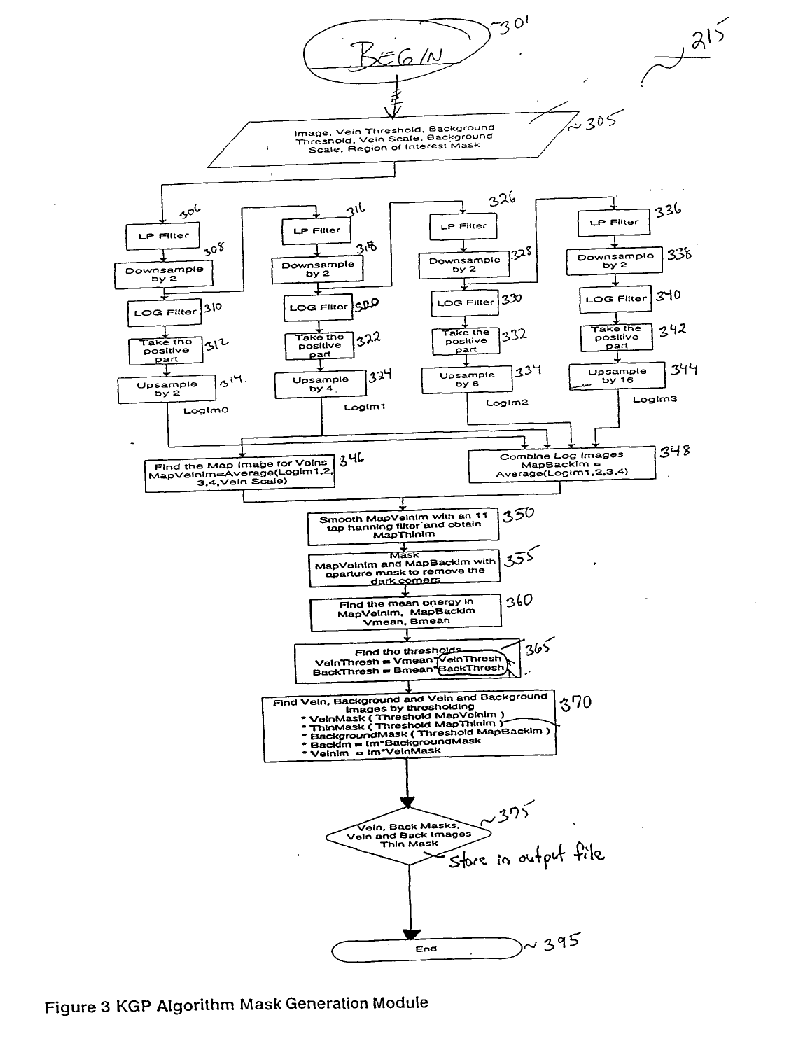

The second part of the Analysis Phase detects and segments the vascular structure within the spectral image from the background area. This is accomplished by using a second order derivative filter referred to as a Laplacian of Gaussian Pyramid (LOG) process. Referring to FIG. 2, this process is implemented at step 215. A more detailed control flow of one embodiment of this process is illustrated in FIG. 3. As shown in steps 306-348, the method of the present invention uses a Log Transform to generate vessel and background area maps (denoted herein as MapVeinIm and MapBackIm, respectively). A Gaussian pyramid is built by blurring and sub-sampling the original image, I(x,y). At each level of the pyramid, a second derivative (Iₓ(x,y) and I_{y}(x,y)) is computed by linear filtering. The Laplacian Image (I²ₓ(x,y) and I²_{y}(x,y)) is computed from the second derivative. The resulting Laplacian image will have non-zero responses at the regions of vascularization and zero responses elsewhere. The Laplacian images are combined at each scale by collapsing the Gaussian pyramid (i.e., blur, up-sample and add at each level).

The combined images are thresholded to create a mask image, M(x,y) (also referred to herein as a vessel or vein mask, V_{Mask}). Values of one in the mask image correspond to regions of vascularization, and values of zero correspond to non-vascular regions. In other words, referring to steps 355-370, vessel and background masks (V_{Mask} and Bₘₐₛₖ) are generated by binarizing the Log image (i.e., MapVeinIm and MapBackIm) using a fixed function of the mean of the Log image, or:

V_{Mask}=M(x,y)=binarize(MapVeinIm,V_{Thresh}) (Eqn. 7)

B_{Mask}=(1−M(x,y))=binarize(MapBackIm, B_{Thresh}) (Eqn. 8)

where,

V_{Thresh}=1.5*V_{Mean} (Eqn. 9)

B_{Thresh}=0.25*B_{Mean} (Eqn. 10)

The vessel and background threshold parameters, 1.5 and 0.25, respectively, are calibration constants loaded at step 105 and depend on the spectral imaging apparatus and other factors, such as the type of imaging (e.g., sublingual or other tissues). In an embodiment, the threshold parameters are experimentally determined numbers. In another embodiment, the threshold parameters are constants determined for the specific instrument type and application (e.g., sublingual Hemoglobin measurement). V_{Mean} and B_{Mean} describe the vessel and background mean energy, respectively, as discussed below.

Referring to FIG. 3, the process for generating the vessel and background masks begins at step 355, where the vessel and background area maps are masked with an aperture mask to remove dark corners. For the sake of brevity, the masked vessel and background area maps will also be denoted herein as MapVeinIm and MapBackIm. At step 360, the mean energy is determined for MapVeinIm and MapBackIm and is denoted as V_{Mean} and Bₘₑₐₙ. At step 365, the vessel and background thresholds are determined as shown by equations 9 and 10. The vessel and background masks are calculated at step 370.

The binary values in the vessel and background masks are used to identify blood vessels in the vascularized region(s). A vessel or vein image, V(x,y), of this region can be created by computing the inner-product of the original intensity image with the mask image, or:

V(x,y)=I(x,y)* M(x,y)=I(x,y)* V_{Mask} (Eqn. 11)

The non-vascular region represents the background structure. A background image, B(x,y), can be created by computing the inner-product of the original intensity image with the inverse of the mask image:

B(x,y)=I(x,y)*(1−M(x,y))=I(x,y)*B_{Mask} (Eqn. 12)

Referring to FIG. 3, the background and vessel images are computed at step 370.

The method of the present invention also calculates a smooth vessel mask, referred to as a thin mask. Referring to FIG. 3, a mapped thin image (denoted herein as MapThinIm) is obtained at step 350 by smoothing the MapVeinIm with an eleven tap hanning filter. The mapped thin image is thresholded at step 370 to calculate the thin mask. The thin mask is based on the same vessel threshold (V_{Tresh}) used to calculate the vessel mask (V_{Mask}). As discussed above, V_{Thresh} can be experimentally determined or depend on the instrument and type of application. As shown at step 225 in FIG. 2, once the thin mask is calculated, a medial axis transformation is applied to the thin mask to find the middle of the vessel segments. This is a one-pixel wide path, which shows the medial axis of the vessels and provides guidance on where to make subsequent measurements of the optical characteristics. At step 230, the thin mask is traced to find the nonzero pixels which indicates the possible measurement locations.

Step 235 involves the generation of an edge gradient image, G_{Image}. The edge gradient image is obtained by a two dimensional convolution of separable prefilter/derivative operators in two passes. The maximum value G. of the absolute edge gradient image is used. The maximum value of the edge gradient image is masked to take edge regions to generate a gradient mask, G_{Mask}. The gradient mask is derived by binarizing G_{MAX} at a mean threshold, or:

G_{Mask}=binarize(G_{MAX}, G_{Thresh}) (Eqn. 13)

where,

G_{Thresh}=2.0*mean(G_{MAX}) (Eqn. 14)

G_{MAX}=max(abs(G_{Image})) (Eqn. 15)

The threshold value, 2.0, is loaded at step 105 and will vary depending on the spectral imaging apparatus and other factors. This process, as illustrated at step 235, improves the diameter estimation occurring at step 240.

In the third part of the Analysis Phase, various geometric properties are calculated and used to screen the spectral images and improve the accuracy of the blood property measurements. These geometric properties include a diameter and area-to-perimeter ratio for the blood vessel of interest. First, the vessel diameter, D, is calculated by generating a diameter mask and using an area based technique to estimate the diameter.

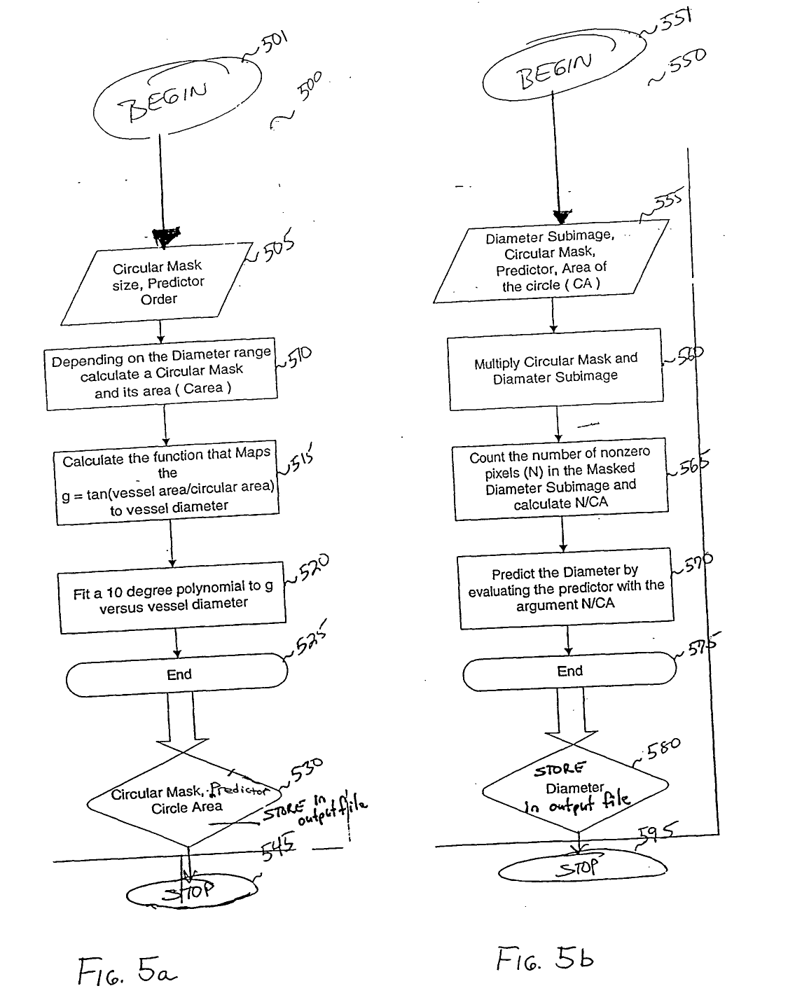

Referring to FIG. 2, at step 240, a diameter mask is generated by adding (logical or) the gradient mask, G_{Mask}, to the vessel mask, V_{Mask} This mask is used in calculating the diameter of the vessels as the algorithm traces along the vessels and makes measurements. FIG. 5A illustrates one embodiment for calculating a circular mask, circular area and diameter predictor parameters used in estimating the vessel diameter, D. FIG. 5B illustrates one embodiment for estimating the vessel diameter, D, from the circular mask, circular area and diameter predictor parameters calculated from the process shown in FIG. 5A.

Referring back to FIG. 2, step 210, a section of the vessel is defined by a binary mask (V_{Mask}). Referring to FIG. 5A, at step 510, a centerline is obtained by skeletonizing the diameter mask using morphological operators. A 2R×2R region of the vessel to be measured is extracted and masked with a circular binary pattern for radius R. This 2R×2R square image is then masked with a circular binary pattern to generate a circular mask. The circular mask would have a “1” inside the circle of radius R and “0” outside of the circle. Measurements are made by counting the number of nonzero entries remaining to calculate the occupied area for the vessel, denoted as Aᵥ. The total possible area for the circular mask is determined by:

A_{c}=πR² (Eqn. 16)

The function g is calculated at step 515 as the tangent of the ratio of Aᵥ to A_{c}, or:

g=tan(Aᵥ/A_{c}) (Eqn. 17)

At step 520, a diameter predictor is generated by fitting a ten degree polynomial to g versus the vessel diameter.

The diameter is estimated by the using an area based technique at step 265. Referring to FIG. 5B, a more detailed control flow is illustrated of one embodiment for determining the diameter estimate. At step 560, the circular mask is multiplied by a diameter subimage to generate a masked diameter subimage. For example, the subimage can be a sub-window of 31×31 pixels. At step 565, the number of nonzero pixels, N, are counted in the masked diameter subimage, denoted as N/A_{c}. The diameter, D, is estimated at step 570 by evaluating the predictor (from step 520) with the argument N/A_{c}.

Another geometric property measured during the Analysis Phase is the vessel's area-to-perimeter ratio. This ratio is calculated at step 245 in FIG. 2. Subjects having low hemoglobin possess a lower number of red blood cells in their blood vessels. For these subjects, vessels are filled with small clumps of red blood cells and a large plasma layer between them. Since the plasma layer is transparent, the vessel walls are very difficult to identify. This is a major problem for the segmentation process. The segmentation process uses the intensity changes or the changes in intensity gradient to segment vessels from the background of the image. For low hemoglobin subjects, this process will segment out clumps of red blood cells instead of the whole vessel. To detect images and subjects suffering from the unfilled vessel effect, the area-to-perimeter ratio is calculated for the vessel mask, V_{Mask}.

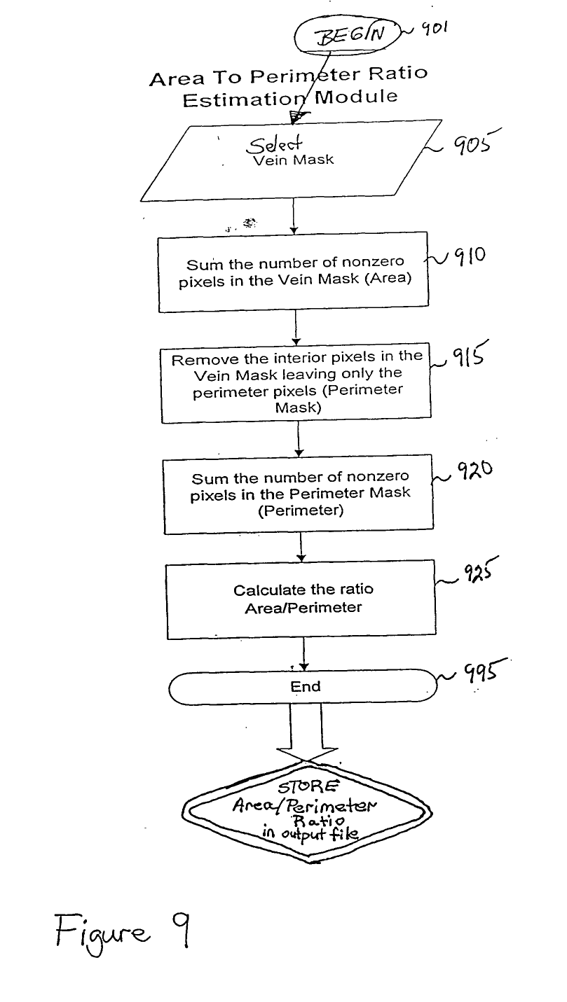

Referring to FIG. 9, a more detailed control flow is illustrated of one embodiment for calculating the area-to-perimeter ratio. At step 905, the vessel mask, V_{Mask}, is retrieved (from the output file create at step 375), and at step 910, the number of nonzero pixels are summed to measure the area, A. At step 915, a perimeter mask is generated by removing the interior pixels in the vessel mask, thereby leaving only the perimeter pixels. The number of nonzero pixels in the perimeter mask are summed at step 920 to measure the perimeter, P. The area-to-perimeter ratio is calculated at step 925 as:

A/P=Area of the Mask÷Perimeter of the Mask (Eqn. 18)

For low hemoglobin subjects, the vessel masks are very clumpy and the area of the vessel mask is considerably smaller. The perimeter of the vessel mask is also much longer for low hemoglobin subjects than for subjects with a higher hemoglobin. Thus, Equation 18 can be used to screen out images having the unfilled vessel effect, as well as determine if the subject has a low hemoglobin concentration. If the subject is determined to have low hemoglobin, the measurements for the small diameter vessels can be rejected during the Calibration and Prediction Phase. For example, the image can be discarded if the diameter estimate is less than 70 microns. The measurements from the larger vessels, e.g. 70 micron or greater, would provide a better estimate of the subjects hemoglobin.

As discussed above in relation to generating the thin mask and gradient mask, measurements are made at those locations exhibiting good measurable properties. This is determined at steps 250-260 and steps 275-280 in FIG. 2 by taking 31×31 pixel windows of the linear segment of the vessel with approximately similar gradient and background on both sides and a background count higher than 300 and less than 1000.

Referring to FIG. 2, at steps 285-290, as each spectral image is processed during the Analysis Phase, the estimated diameter, D, and the optical density (or contrast of the vessel), denoted as OD or K, are determined and stored in an output file (as also shown at step 155). The optical density is calculated from the mean of the vessel image V(x,y), denoted as I,, and the mean of the background image B(x,y), denoted as I_{b}. For example, the optical density can be measured by:

K=log₁₀(I_{b}÷Iᵥ) (Eqn. 19)

As shown at step 250, the diameter and optical density are calculated for each nonzero pixel in the thin mask. Afterwards, at step 292, contrast value is normalized and averaged as:

L=K÷D (Eqn. 20)

This value is also stored to the output file, at step 155, for use in the Calibration and Prediction Phase.

IV. Calibration & Prediction Phase

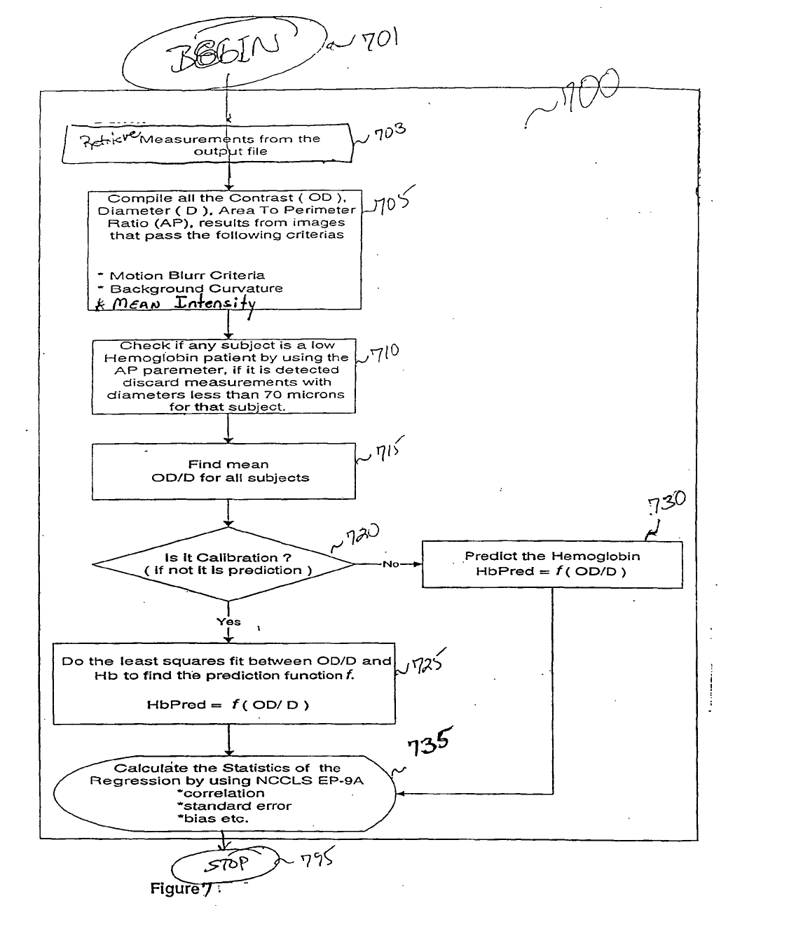

The Calibration and Prediction Phase processes the values obtained from the Screening and Analysis Phases, namely the mean image intensity, motion blur parameters, background curvature, optical density or contrast, diameter and area-to-perimeter ratio. FIG. 7 illustrates one embodiment of a control flow for implementing the Calibration and Prediction Phase. At step 703, the recorded measurements are retrieved from the output file and, at step 705, spectral images having background curvature, motion blur, and mean intensity exceeding predetermined threshold values can be discarded, as discussed above, to select the images having better quality. At step 710, the spectral image is screened to detect low hemoglobin as discussed in reference to step 245. If low hemoglobin is detected, images having small diameter estimates, e.g. less than 70 microns, can be discarded.

At step 715, the normalized optical density, L, is taken for each spectral image that passes the screening tests in steps 705 and 710. Step 715 also determines the average value of the normalized optical density for all images from the subject.

The normalized optical density is used to measure the hemoglobin in the subject because L is functionally related to the hemoglobin and can be expressed as:

Hb=aL÷b (Eqn. 21)

where a and b are calibration constants that can be determined empirically. For example, referring to steps 720 and 725, the calibration constants can be determined by performing a least squares fit between L and the actual or known Hb of the subject. Once the constants are determined, the hemoglobin can be estimated by using Equation 20 at step 730. As can be seen, equation 22 shows a linear relationship between Hb and L. However, Hb can also be represented as a nonlinear function of L.

At step 735, the NCCLSEP-9A protocol is used to measure the agreement between two analytic measurement methods for clinical chemistry devices. In an embodiment, the protocol is used to calculate the amount of agreement between the Hemoglobin measured at step 725 or step 730 versus the standard Coulter method. Step 735 is optional and provides a prediction function that gives the best estimate of the Hemoglobin from the measurements.

In another embodiment, steps 720, 725 and 735 are omitted. In this embodiment, an optimal predictor function is implemented at step 730 that is based on the instrument and data acquisition method. Hence, the calibration step (EP-9A calculations) will not be implemented, and the measurements would only provide a prediction.

As is apparent from the foregoing description, the present invention was developed primarily to analyze blood components in a non-invasive manner. However, it will be clear to persons skilled in the relevant art(s) that the analysis techniques of this invention have utility beyond the medical applications described above. The invention has application outside the medical area and can be used generally to analyze visualizable components in a fluid flowing in any vascular system, such as a tube, the walls of which are transparent to transmitted and reflected light.

The present invention can be implemented using hardware, software or a combination thereof and can be implemented in one or more computer systems or other processing systems. In fact, in one embodiment, the invention is directed toward one or more computer systems capable of carrying out the functionality described herein.

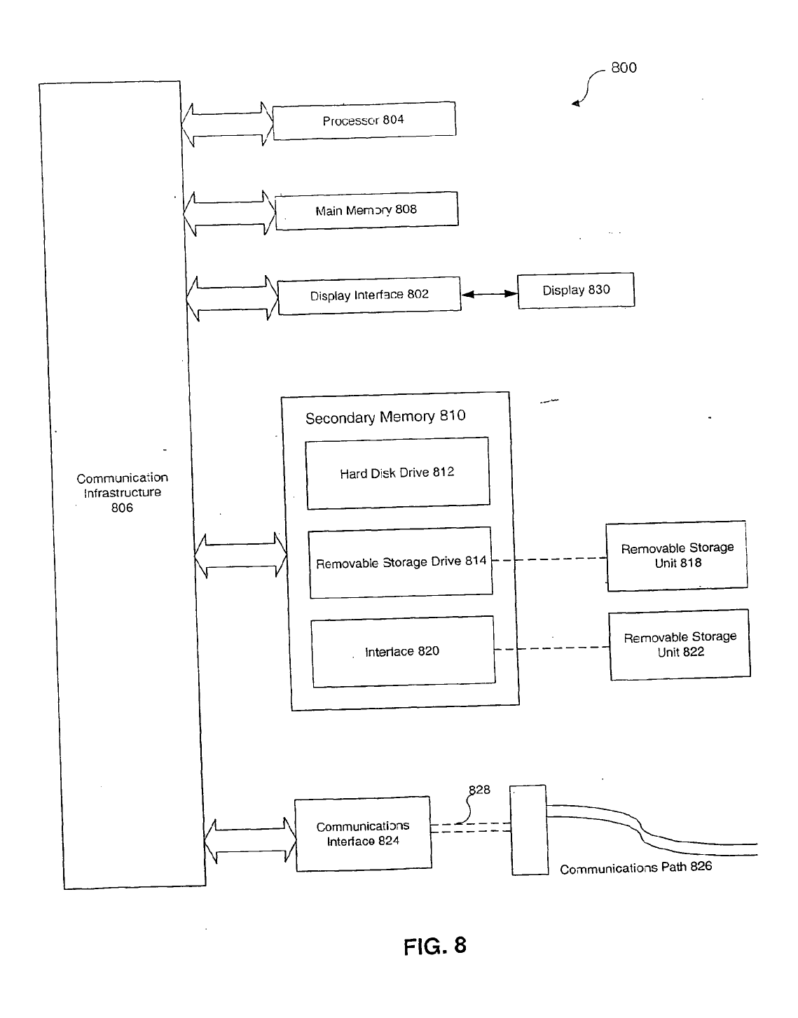

An exemplary screening, analyzing, and calibration and prediction means for use in the present invention is shown as computer system 800 in FIG. 8. Computer system 800 includes one or more processors, such as processor 804. The processor 804 is connected to a communication infrastructure 806 (e.g., a communications bus, cross-overbar, or network). Various software embodiments are described in terms of this exemplary computer system. After reading this description, it will become apparent to a person skilled in the relevant art(s) how to implement the invention using other computer systems and/or computer architectures.

Computer system 800 can include a display interface 802 that forwards graphics, text, and other data from the communication infrastructure 806 (or from a frame buffer not shown) for display on the display unit 830.

Computer system 800 also includes a main memory 808, preferably random access memory (RAM), and can also include a secondary memory 810. The secondary memory 810 can include, for example, a hard disk drive 812 and/or a removable storage drive 814, representing a floppy disk drive, a magnetic tape drive, an optical disk drive, etc. The removable storage drive 814 reads from and/or writes to a removable storage unit 818 in a well-known manner. Removable storage unit 818, represents a floppy disk, magnetic tape, optical disk, etc. which is read by and written to removable storage drive 814. As will be appreciated, the removable storage unit 818 includes a computer usable storage medium having stored therein computer software and/or data.

In alternative embodiments, secondary memory 810 can include other similar means for allowing computer programs or other instructions to be loaded into computer system 800. Such means can include, for example, a removable storage unit 822 and an interface 820. Examples of such can include a program cartridge and cartridge interface (such as that found in video game devices), a removable memory chip (such as an EPROM, or PROM) and associated socket, and other removable storage units 822 and interfaces 820 which allow software and data to be transferred from the removable storage unit 822 to computer system 800.

Computer system 800 can also include a communications interface 824. Communications interface 824 allows software and data to be transferred between computer system 800 and external devices. Examples of communications interface 824 can include a modem, a network interface (such as an Ethernet card), a communications port, a PCMCIA slot and card, etc. Software and data transferred via communications interface 824 are in the form of signals 828 which can be electronic, electromagnetic, optical or other signals capable of being received by communications interface 824. These signals 828 are provided to communications interface 824 via a communications path (i.e., channel) 826. This channel 826 carries signals 828 and can be implemented using wire or cable, fiber optics, a phone line, a cellular phone link, an RF link and other communications channels.

In this document, the terms “computer program medium” and “computer usable medium” are used to generally refer to media such as removable storage drive 814, a hard disk installed in hard disk drive 812, and signals 828. These computer program products are means for providing software to computer system 800. The invention is directed to such computer program products.

Computer programs (also called computer control logic) are stored in main memory 808 and/or secondary memory 810. Computer programs can also be received via communications interface 824. Such computer programs, when executed, enable the computer system 800 to perform the features of the present invention as discussed herein. In particular, the computer programs, when executed, enable the processor 804 to perform the features of the present invention. Accordingly, such computer programs represent controllers of the computer system 800.

In an embodiment where the invention is implemented using software, the software can be stored in a computer program product and loaded into computer system 800 using removable storage drive 814, hard drive 812 or communications interface 824. The control logic (software), when executed by the processor 804, causes the processor 804 to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to persons skilled in the relevant art(s).

In yet another embodiment, the invention is implemented using a combination of both hardware and software.

V. Conclusion

While various embodiments of the present invention have been described above, it should be understood that they have been presented by way of example, and not limitation. It will be apparent to persons skilled in the relevant art(s) that various changes in form and detail can be made therein without departing from the spirit and scope of the invention. Thus, the present invention should not be limited by any of the above described exemplary embodiments.

What is claimed is:

1. A method for analyzing visualizable components in a fluid flowing in a vascular system, the walls of which are substantially transparent to transmitted and reflected light, using a light transmitting device that is capable of transmitting light through the walls of a region of the vascular system into a sub-surface region thereof, an image capturing device capable of capturing images reflected from the sub-surface region of the vascular system illuminated by the light transmitting device to create a spectral image, and a processing unit in conmmunication with the image capturing device, comprising the steps of:
(a) receiving in the processing unit a reflected spectral image of the subsurface region captured by the image capturing device;
(b) screening said reflected spectral image to determine whether at least one of a plurality of properties of said reflected spectral image exceeds a predetermined threshold;
(c) analyzing said reflected spectral image to measure a background curvature of the vascular system in the region of the vascular system through which light is transmitted;
(d) analyzing said reflected spectral image to segment the vascular and non-vascular regions within said reflected spectral image; and
(e) measuring an optical density of the vascular region to calculate a contrast value between the vascular and nonvascular regions within said reflected spectral image.

2. The method according to claim 1, wherein the fluid comprises blood flowing in a blood vessel of a mammalian vascular system, and the visualizable components comprise blood components, including red blood cells, white blood cells, platelets, etc., and wherein:
step (a) comprises receiving in the processing unit a reflected spectral image of the blood components in the region of the vascular system through which the light is transmitted and reflected; and
step (c) comprises analyzing said reflected spectral image to measure the background curvature of the region of the blood vessel through which light is transmitted.

3. The method according to claim 1, wherein step (e) comprises the step of adding a calibration constant to said contrast value.

4. The method according to claim 1, wherein step (d) comprises the steps of:
(i) generating a vessel image to identify the vascular regions of said reflected spectral image and a background image to identify the non-vascular regions of said reflected spectral image;
(ii) generating a vessel diameter estimate of said vessel image; and
(iii) generating an area-to-perimeter ratio of the vascular regions.

5. The method according to claim 4, further comprising the step of calculating a smooth vessel image to generate a thin image.

6. The method according to claim 4, further comprising the steps of:
(iv) binarizing a log transform of said reflected spectral image to generate a vessel mask and a background mask;
(v) computing the inner-product of the intensity of said reflected spectral image with said vessel mask to generate said vessel image; and
(vi) computing the inner product of the intensity of said reflected spectral image with said background mask to generate said background image.

7. The method according to claim 6, further comprising the steps of:
(vii) generating a gradient mask;
(viii) adding said gradient mask to said vessel mask to generate a diameter mask; and
(ix) generating said diameter estimate from said diameter mask.

8. The method according to claim 4, further comprising the step of generating a binary circular mask, wherein said diameter estimate is a function of said binary circular mask.

9. The method according to claim 4, further comprising the steps of:
(iv) generating a vessel contrast from a mean vessel image and a mean background image; and
(v) generating a normalized contrast value from said diameter estimate and said vessel contrast.

10. The method according to claim 1, wherein said at least one of a plurality of properties is selected from a group consisting of a slope ratio, a mean image intensity, a motion blur, a background curvature and an area-to-perimeter ratio.

11. The method according to claim 10, wherein said area-to-perimeter ratio is used to detect a subject having a low hemoglobin concentration.

12. An apparatus for analyzing visualizable components in a fluid flowing in a vascular system, the walls of which are substantially transparent to transmitted and reflected light, comprising:
a light transmitting device for transmitting light through the walls of a region of the vascular system into a sub-surface region thereof;
an image capturing device for capturing images reflected from the sub-surface region of the vascular system illuminated by said light transmitting device to create a spectral image; and
a processing unit in communication with said image capturing device and comprising:
receiving means for receiving a reflected spectral image of the subsurface region captured by said image capturing device,
screening means for screening said reflected spectral image to determine whether at least one of a plurality of properties of said reflected spectral image exceeds a predetermined threshold,
first analyzing means for analyzing said reflected spectral image to measure a background curvature of the vascular system in the region of the vascular system through which light is transmitted,
second analyzing means for analyzing said reflected spectral image to segment the vascular and non-vascular regions within said reflected spectral image, and
third analyzing means for measuring an optical density of the vascular region to calculate a contrast value between the vascular and non-vascular regions within said reflected spectral image.

13. The apparatus according to claim 12, wherein the fluid comprises blood flowing in a blood vessel of a mammalian vascular system, and the visualizable components comprise blood components, including red blood cells, white blood cells, platelets, etc., and wherein:
said receiving means comprises means for receiving in the processing unit a reflected spectral image of the blood components in the region of the vascular system through which the light is transmitted and reflected; and
said first analyzing means comprises means for analyzing said reflected spectral image to measure the background curvature of the region of the blood vessel through which light is transmitted.

14. The apparatus according to claim 12, wherein said third analyzing means comprises means for adding a calibration constant to said contrast value.

15. The apparatus according to claim 12, wherein said second analyzing means comprises:
first generating means for generating a vessel image to identify the vascular regions of said reflected spectral image and a background image to identify the non-vascular regions of said reflected spectral image;
second generating means for generating a vessel diameter estimate of said vessel image; and
third generating means for generating an area-to-perimeter ratio of the vascular regions.

16. The apparatus according to claim 15, further comprising calculating means for calculating a smooth vessel image to generate a thin image.

17. The apparatus according to claim 15, further comprising:
fourth generating-means for binarizing a log transform of said image to generate a vessel mask and a background mask;
first computing means for computing the inner-product of the intensity of said reflected spectral image with said vessel mask to generate said vessel image; and
second computing means for computing the inner product of the intensity of said reflected spectral image with said background mask to generate said background image.

18. The apparatus according to claim 17, further comprising:
fifth generating means for generating a gradient mask and adding said gradient mask to said vessel mask to generate a diameter mask; and
sixth generating means for generating said diameter estimate from said diameter mask.

19. The apparatus according to claim 15, further comprising:
fourth generating means for generating a binary circular mask, wherein said diameter estimate is a function of said binary circular mask.

20. The apparatus according to claim 15, further comprising:
fourth generating means for generating a vessel contrast from a mean vessel image and a mean background image; and
fifth generating means for generating a normalized contrast value from said diameter estimate and said vessel contrast.

21. The apparatus according to claim 12, wherein said at least one of a plurality of properties is selected from a group consisting of a slope ratio, a mean image intensity, a motion blur, a background curvature and an area-to-perimeter ratio.

22. The apparatus according to claim 21, wherein said area-to-perimeter ratio is used to detect a subject having a low hemoglobin concentration.

23. A computer program product comprising a computer useable medium having computer program logic recorded thereon for analyzing visualizable components in a fluid flowing in a vascular system, the walls of which are substantially transparent to transmitted and reflected light, using a light transmitting device that is capable of transmitting light through the walls of a region of the vascular system into a sub-surface region thereof, an image capturing device capable of capturing images reflected from the sub-surface region of the vascular system illuminated by the light transmitting device to create a spectral image, and a processing unit in communication with the image capturing device, said computer program logic comprising:
first computer program product means for receiving in the processing unit a reflected spectral image of the subsurface region captured by the image capturing device;
second program product means for screening said reflected spectral image to determine whether at least one of a plurality of properties of said reflected spectral image exceeds a predetermined threshold;
third program product means for analyzing said reflected spectral image to measure a background curvature of the vascular system in the region of the vascular system through which light is transmitted;
fourth program product means for analyzing said reflected spectral image to segment the vascular and non-vascular regions within said reflected spectral image; and
fifth program product means for measuring an optical density of the vascular region to calculate a contrast value between the vascular and non-vascular regions within said reflected spectral image.

24. A computer program product according to claim 23, wherein the fluid comprises blood flowing in a blood vessel of a mammalian vascular system, and the visualizable components comprise blood components, including red blood cells, white blood cells, platelets, etc., and wherein:
said first computer program product means comprises computer program product means for receiving in the processing unit a reflected spectral image of the blood components in the region of the vascular system through which the light is transmitted and reflected; and
said third computer program product means comprises computer program product means for analyzing said reflected spectral image to measure the background curvature of the region of the blood vessel through which light is transmitted.
